(19) United States
(12) Patent Application Publication
(10) Pub. No.: US 2024/0041938 A1
(43) Pub. Date: Feb. 8, 2024

(54) PREVENTION OF SPACE TRAVEL ASSOCIATED BONE DENSITY LOSS BY REGENERATIVE CELL POPULATIONS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US); Timothy WARBINGTON, Phoenix, AZ (US)

(73) Assignee: CREATIVE MEDICAL TECHNOLOGIES, INC., Phoenix, AZ (US)

(21) Appl. No.: 18/366,539

(22) Filed: Aug. 7, 2023

Related U.S. Application Data
(60) Provisional application No. 63/395,834, filed on Aug. 7, 2022.

(51) Int. Cl. A61K 35/28; A61K 35/545; A61K 35/50; A61K 35/35; A61K 35/30; A61K 35/51; A61K 35/32; A61K 35/14

(52) U.S. Cl. CPC A61K 35/28; A61K 35/545; A61K 35/50; A61K 35/35; A61K 35/30; A61K 35/51; A61K 35/32; A61K 35/14

(57) ABSTRACT

Disclosed are means, methods and compositions of matter useful for prevention of space flight associated bone density loss. In one embodiment, the invention provides cellular populations capable of suppressing osteoclast function and/or stimulating osteoblast activity. The use of mesenchymal stem cell populations alone, or more preferably, selected by expression of CD56 protein are disclosed. Cells may be administered systemically, or locally through the use of various matrices disclosed in this invention.

### CROSS-REFERENCE TO RELATED APPLICATIONS

This application claims the benefit of priority to U.S. Provisional Application No. 63/395,834, titled “Prevention of Space Travel Associated Bone Density Loss by Regenerative Cell Populations” filed Aug. 7, 2022, which is incorporated herein by reference in its entirety

### FIELD OF THE INVENTION

The teachings herein relate to the use of regenerative cells for preventing and treating bone density loss through space travel.

### BACKGROUND

Numerous studies have shown that space travel unfortunately causes multiple deleterious effects on health. Negative impact of space travel has been documented on immune system effects, accelerated aging, muscle atrophy, bone loss, balance, cardiac atrophy and other cardiovascular effects, and increased susceptibility to infection and/or reactivation of latent infection, this was published in Crucian et al. (2018), Immune System Dysregulation During Spaceflight: Potential Countermeasures for Deep Space Exploration Missions, Front. Immunol. 9: 1437; Perhonen et al., Additionally, cardiac atrophy after bed rest and spaceflight was published in J Appl Physiol (1985). 2001 August;91(2):645-53. doi: PMID: 11457776; https://phys.org/news/2020-10-human-heart-space-mathematical.ht-ml; Mermel (2013), Infection Prevention and Control During Prolonged Human Space Travel, Clinical Infectious Diseases 56(1): 123-130. It is believed that some of the adverse effects are due to the effects of microgravity and/or exposure to space radiation.

Inflammation has been associated with spaceflight. This has been reported from the perspective that NF-.kappa.B was elevated in astronauts after short-duration spaceflight, which was published in Zwart et al. (2010), Capacity of Omega-3 Fatty Acids or Eicosapentaenoic Acid to Counteract Weightlessness-Induced Bone Loss by Inhibiting NF-.kappa.B Activation: From Cells to Bed Rest to Astronauts, American Society for Bone and Mineral Research, 25(5): 1049-1057; the contents of which are expressly incorporated by reference herein. Specifically, Zwart showed that NF-.kappa.B p65 expression was increased by almost 500% in space shuttle crew members. NF-.kappa.B is a transcriptional activator that induces transcription factors that play a role in inflammation, muscle atrophy, and bone resorption. Zwart also provided evidence that the omega-3 fatty acid and eicosapentaenoic acid, can decrease NF-.kappa.B activation in modeled weightlessness and suggested that inhibition of this transcriptional activator can mitigate the spaceflight-associated effects on bone, muscle, and immune function. Despite the recognition in the art of the numerous negative health effects associated with spaceflight, there remains a need in the art to mitigate or treat such effects.

### SUMMARY

Preferred embodiments include methods of preventing low gravity or zero gravity associated bone density loss comprising of administration of a regenerative cell population.

Preferred methods include embodiments wherein the low gravity environment is a low planetary orbit, an interplanetary voyage or inhabiting a planet or moon with gravity less than 1 G.

Preferred methods include embodiments wherein the low planetary orbit is selected from low Earth orbit, low Moon orbit or low Mars orbit.

Preferred methods include embodiments wherein said bone density loss is associated with reduction in osteoblast activity.

Preferred methods include embodiments wherein said bone density loss is associated with enhancement in osteoclast activity.

Preferred methods include embodiments wherein said bone density loss is associated with increased levels of TNF alpha.

Preferred methods include embodiments wherein said TNF-alpha is found in peripheral blood.

Preferred methods include embodiments wherein said TNF-alpha is found in the bone microenvironment.

Preferred methods include embodiments wherein said TNF-alpha is found in the osteoblasts.

Preferred methods include embodiments wherein said increase osteoclast activity is associated with enhanced RANK ligand activity as compared to an age-matched healthy control.

Preferred methods include embodiments wherein said RANK ligand is found in peripheral blood.

Preferred methods include embodiments wherein said RANK ligand is found in the bone microenvironment.

Preferred methods include embodiments wherein said RANK ligand is found in the osteoblasts.

Preferred methods include embodiments wherein said increase osteoclast activity is associated with enhanced interleukin-1 beta activity as compared to an age-matched healthy control.

Preferred methods include embodiments wherein said interleukin-1 beta is found in peripheral blood.

Preferred methods include embodiments wherein said interleukin-1 beta is found in the bone microenvironment.

Preferred methods include embodiments wherein said interleukin-1 beta is found in the osteoblasts.

Preferred methods include embodiments wherein said increased osteoclast activity is associated with enhanced interleukin-6 activity as compared to an age-matched healthy control.

Preferred methods include embodiments wherein said interleukin-6 is found in peripheral blood.

Preferred methods include embodiments wherein said interleukin-6 is found in the bone microenvironment.

Preferred methods include embodiments wherein said interleukin-6 is found in the osteoblasts.

Preferred methods include embodiments wherein said increased osteoclast activity is associated with enhanced interleukin-8 activity as compared to an age-matched healthy control.

Preferred methods include embodiments wherein said interleukin-8 is found in peripheral blood.

Preferred methods include embodiments wherein said interleukin-8 is found in the bone microenvironment.

Preferred methods include embodiments wherein said interleukin-8 is found in the osteoblasts.

Preferred methods include embodiments wherein said increased osteoclast activity is associated with enhanced interleukin-11 activity as compared to an age-matched healthy control.

Preferred methods include embodiments wherein said interleukin-11 is found in peripheral blood.

Preferred methods include embodiments wherein said interleukin-11 is found in the bone microenvironment.

Preferred methods include embodiments wherein said interleukin-11 is found in the osteoblasts.

Preferred methods include embodiments wherein said increased osteoclast activity is associated with enhanced interleukin-15 activity as compared to an age-matched healthy control.

Preferred methods include embodiments wherein said interleukin-15 is found in peripheral blood.

Preferred methods include embodiments wherein said interleukin-15 is found in the bone microenvironment.

Preferred methods include embodiments wherein said interleukin-15 is found in the osteoblasts.

Preferred methods include embodiments wherein said increased osteoclast activity is associated with enhanced interleukin-17 activity as compared to an age-matched healthy control.

Preferred methods include embodiments wherein said interleukin-17 is found in peripheral blood.

Preferred methods include embodiments wherein said interleukin-17 is found in the bone microenvironment.

Preferred methods include embodiments wherein said interleukin-17 is found in the osteoblasts.

Preferred methods include embodiments wherein said increased osteoclast activity is associated with enhanced interleukin-18 activity as compared to an age-matched healthy control.

Preferred methods include embodiments wherein said interleukin-18 is found in peripheral blood.

Preferred methods include embodiments wherein said interleukin-18 is found in the bone microenvironment.

Preferred methods include embodiments wherein said interleukin-18 is found in the osteoblasts.

Preferred methods include embodiments wherein said increased osteoclast activity is associated with enhanced interleukin-33 activity as compared to an age-matched healthy control.

Preferred methods include embodiments wherein said interleukin-33 is found in peripheral blood.

Preferred methods include embodiments wherein said interleukin-33 is found in the bone microenvironment.

Preferred methods include embodiments wherein said interleukin-33 is found in the osteoblasts.

Preferred methods include embodiments wherein said regenerative cell is selected from either alone or in combination from a group comprising of: stem cells, committed progenitor cells, and differentiated cells.

Preferred methods include embodiments wherein said stem cells are selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Preferred methods include embodiments wherein said embryonic stem cells are totipotent.

Preferred methods include embodiments wherein said embryonic stem cells express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred methods include embodiments wherein said cord blood stem cells are multipotent and capable of differentiating into endothelial, smooth muscle, and neuronal cells.

Preferred methods include embodiments wherein said cord blood stem cells are identified based on expression of one or more antigens selected from a group comprising: SSEA-3, SSEA-4, CD9, CD34, c-kit, OCT-4, Nanog, and CXCR-4.

Preferred methods include embodiments wherein said cord blood stem cells do not express one or more markers selected from a group comprising of: CD3, CD34, CD45, and CD11b.

Preferred methods include embodiments wherein said placental stem cells are isolated from the placental structure.

Preferred methods include embodiments wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

Preferred methods include embodiments wherein said bone marrow stem cells comprise of bone marrow mononuclear cells.

Preferred methods include embodiments wherein said bone marrow stem cells are selected based on the ability to differentiate into one or more of the following cell types: endothelial cells, smooth muscle cells, and neuronal cells.

Preferred methods include embodiments wherein said bone marrow stem cells are selected based on expression of one or more of the following antigens: CD34, c-kit, flk-1, Stro-1, CD105, CD73, CD31, CD146, vascular endothelial-cadherin, CD133 and CXCR-4.

Preferred methods include embodiments wherein said bone marrow stem cells are enriched for expression of CD133.

Preferred methods include embodiments wherein said amniotic fluid stem cells are isolated by introduction of a fluid extraction means into the amniotic cavity under ultrasound guidance.

Preferred methods include embodiments wherein said amniotic fluid stem cells are selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1.

Preferred methods include embodiments wherein said amniotic fluid stem cells are selected based on lack of expression of one or more of the following antigens: CD34, CD45, and HLA Class II.

Preferred methods include embodiments wherein said neuronal stem cells are selected based on expression of one or more of the following antigens: RC-2, 3CB2, BLB, Sox-2hh, GLAST, Pax 6, nestin, Muashi-1, NCAM, A2B5 and prominin.

Preferred methods include embodiments wherein said circulating peripheral blood stem cells are characterized by the ability to proliferate in vitro for a period of over 3 months.

Preferred methods include embodiments wherein said circulating peripheral blood stem cells are characterized by expression of CD34, CXCR4, CD117, CD113, and c-met.

Preferred methods include embodiments wherein said circulating peripheral blood stem cells lack substantial expression of differentiation associated markers.

Preferred methods include embodiments wherein said differentiation of associated markers are selected from a group comprising of: CD2, CD3, CD4, CD11, CD11a, Mac-1, CD14, CD16, CD19, CD24, CD33, CD36, CD38, CD45, CD56, CD64, CD68, CD86, CD66b, and HLA-DR.

Preferred methods include embodiments wherein said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein said mesenchymal stem cells do not express substantial levels of HLA-DR, CD117, and CD45.

Preferred methods include embodiments wherein said mesenchymal stem cells are derived from a group selected of: bone marrow, adipose tissue, umbilical cord blood, placental tissue, peripheral blood mononuclear cells, differentiated embryonic stem cells, and differentiated progenitor cells.

Preferred methods include embodiments wherein said germinal stem cells express markers selected from a group comprising of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1.

Preferred methods include embodiments wherein said adipose tissue derived stem cells express markers selected from a group comprising of CD13, CD29, CD44, CD63, CD73, CD90, CD166, Aldehyde dehydrogenase (ALDH), and ABCG2.

Preferred methods include embodiments wherein said adipose tissue derived stem cells are a population of purified mononuclear cells extracted from adipose tissue capable of proliferating in culture for more than 1 month.

Preferred methods include embodiments wherein said exfoliated teeth derived stem cells express markers selected from a group comprising of: STRO-1, CD146 (MUC18), alkaline phosphatase, MEPE, and bFGF.

Preferred methods include embodiments wherein said hair follicle stem cells express markers selected from a group comprising of: cytokeratin 15, Nanog, and Oct-4.

Preferred methods include embodiments wherein said hair follicle stem cells are capable of proliferating in culture for a period of at least one month.

Preferred methods include embodiments wherein said hair follicle stem cells secrete one or more of the following proteins when grown in culture: basic fibroblast growth factor (bFGF), endothelin-1 (ET-1) and stem cell factor (SCF).

Preferred methods include embodiments wherein said dermal stem cells express markers selected from a group comprising of: CD44, CD13, CD29, CD90, and CD105.

Preferred methods include embodiments wherein said dermal stem cells are capable of proliferating in culture for a period of at least one month.

Preferred methods include embodiments wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred methods include embodiments wherein said reprogrammed stem cells are selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

Preferred methods include embodiments wherein said nuclear transfer comprises introducing nuclear material to a cell substantially enucleated, said nuclear material deriving from a host whose genetic profile is sought to be dedifferentiated.

Preferred methods include embodiments wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Preferred methods include embodiments wherein said DNA demethylating agent is selected from a group comprising of: 5-azacytidine, psammaplin A, and zebularine.

Preferred methods include embodiments wherein said histone deacetylase inhibitor is selected from a group comprising of: valproic acid, trichostatin-A, trapoxin A and depsipeptide.

The side population cells of claim 46, wherein said cells are identified based on expression multidrug resistance transport protein (ABCG2) or ability to efflux intracellular dyes such as rhodamine-123 and or Hoechst 33342.

The side population cells of claim 85, wherein said cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

Preferred methods include embodiments wherein said committed progenitor cells are selected from a group comprising of: endothelial progenitor cells, neuronal progenitor cells, and hematopoietic progenitor cells.

Preferred methods include embodiments wherein said committed endothelial progenitor cells are purified from the bone marrow.

Preferred methods include embodiments wherein said committed endothelial progenitor cells are purified from peripheral blood.

Preferred methods include embodiments wherein said committed endothelial progenitor cells are purified from peripheral blood of a patient whose committed endothelial progenitor cells are mobilized by administration of a mobilizing agent or therapy.

Preferred methods include embodiments wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG-CoA reductase inhibitors and small molecule antagonists of SDF-1.

Preferred methods include embodiments wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Preferred methods include embodiments wherein said committed endothelial progenitor cells express markers selected from a group comprising of: CD31, CD34, AC133, CD146 and flk1.

Preferred methods include embodiments wherein said committed hematopoietic cells are purified from the bone marrow.

Preferred methods include embodiments wherein said committed hematopoietic progenitor cells are purified from peripheral blood.

Preferred methods include embodiments wherein said committed hematopoietic progenitor cells are purified from peripheral blood of a patient whose committed hematopoietic progenitor cells are mobilized by administration of a mobilizing agent or therapy.

Preferred methods include embodiments wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG-CoA reductase inhibitors and small molecule antagonists of SDF-1.

Preferred methods include embodiments wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Preferred methods include embodiments wherein said committed hematopoietic progenitor cells express the marker CD133.

Preferred methods include embodiments wherein said committed hematopoietic progenitor cells express the marker CD34.

The method of claims 1, wherein an antioxidant is administered at a therapeutically sufficient concentration to a patient in need thereof.

Preferred methods include embodiments wherein said antioxidant is selected from a group comprising of: ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol.

Preferred methods include embodiments wherein an NF-kappa B inhibitor is administered prior to, subsequently with, or after administration of regenerative cells.

Preferred methods include embodiments wherein said NF-kappa B inhibitor is administered directly into the ovary.

Preferred methods include embodiments wherein said NF-kappa B inhibitor is administered systemically.

Preferred methods include embodiments wherein said NF-kappa B inhibitor is administered using bone targeting technology.

Preferred methods include embodiments wherein said bone targeting technology are liposomes.

Preferred methods include embodiments wherein said bone targeting technology are immunoliposomes.

Preferred methods include embodiments wherein said bone targeting technology are nanoparticles.

Preferred methods include embodiments wherein said bone targeting technology are quantum dots.

Preferred methods include embodiments wherein said regenerative cells are used to treat the bone loss and radiation sickness outside of the earth's ozone layer is also caused by exposure to particles trapped in the Earth's magnetic field.

Preferred methods include embodiments wherein said regenerative cells are used to treat the bone loss and radiation sickness outside of the earth's ozone layer is also caused by exposure to particles shot into space during solar flares (solar particle events).

Preferred methods include embodiments wherein said regenerative cells are used to treat the bone loss and radiation sickness outside of the earth's ozone layer is also caused by exposure to galactic cosmic rays.

Preferred methods include embodiments wherein the regenerative cells also protect the bone marrow from the exposure to particles trapped in the Earth's magnetic field.

Preferred methods include embodiments wherein the regenerative cells also protect the bone marrow from the exposure to particles shot into space during solar flares (solar particle events).

Preferred methods include embodiments wherein the regenerative cells also protect the bone marrow from the exposure to galactic cosmic rays.

### DETAILED DESCRIPTION OF THE INVENTION

The invention provides novel means of protecting a mammal from space travel induced bone loss through administration of regenerative cells and/or inhibitors of NF-kappa. In one embodiment, umbilical cord mesenchymal stem cells are administered together with inhibitors of NF-kappa B selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2C1), Anethole, Baoganning, Black raspberry extracts (cyanidin 3-O-glucoside, cyanidin 3-O-(2(G)-xylosylrutinoside), cyanidin 3-0-rutinoside), Buddlejasaponin IV, Cacospongionolide B, Calagualine, Carbon monoxide, Cardamonin, Cycloepoxydon; 1-hydroxy-2-hydroxymethyl-3-pent-l-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic). In some embodiments, anti-inflammatory agents are also utilized as a regenerative adjuvant.

As used herein and in the appended claims, the singular forms “a,” “an,” and “the” include plural referents unless the context clearly dictates otherwise. Thus, for example, reference to “an oligosaccharide” includes a plurality of such oligosaccharides and reference to “the therapeutic agent” includes reference to one or more therapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs.

The term “spaceflight” means travel outside of the Earth's atmosphere, for example on the Space Shuttle, the International Space Station, a satellite, a rocket, or other space vehicle, such that microgravity conditions exist. Spaceflight includes travel in Earth's orbit, as well as travel through space, such as between planets.

“Microgravity” is a state in which there is very little net gravitational force, for example, gravity less than about 1 G. Microgravity conditions exist in space, for example, aboard the Space Shuttle, the International Space Station, a satellite, or a rocket while in flight outside the Earth's atmosphere. Simulated microgravity is microgravity which is simulated by a set of Earth-based conditions that mimic microgravity, such as by balancing gravity with equal and opposite forces (for example, shear force, centripetal force, Coriolis forces, buoyancy, and/or magnetic field). In one example, simulated microgravity may be generated by use of a clinostat, such as a rotating wall vessel (RWV). In another example, simulated microgravity may be generated by a random positioning machine (RPM). The terms “microgravity conditions” and “microgravity” are used synonymously herein. Normal gravity is the gravity normally experienced on Earth, such as on the surface of the Earth and/or in its atmosphere (for example, in aircraft in the atmosphere of the Earth). Gravity is measured in terms of acceleration due to gravity, denoted by g. The strength (or apparent strength) of Earth's gravity varies with latitude, altitude, local topography, and geology. In some examples, normal gravity (such as 1G) is about 9-10 m/s.sup.2, for example, about 9.7-9.9 m/s.sup.2. In particular, preferred embodiments, normal gravity is that experienced on the surface of the Earth under normal gravity at that location on the Earth. The terms “microgravity” and “low gravity” are used interchangeably herein.

A “low gravity environment” is an environment of low gravity or microgravity as described herein. Examples of a low gravity environment include a low planetary orbit (e.g., low Earth orbit, low Moon orbit or low Mars orbit), an interplanetary voyage or inhabiting a planet or moon with gravity less than 1 G.

The terms “spaceflight associated disease or condition” and “spaceflight induced disease or condition,” and the like include diseases, disorders and conditions that occur during spaceflight or in a low gravity environment including, but not limited to, those associated with the effects of microgravity (or low gravity) and/or spaceflight associated radiation exposure. Non-limiting examples of such conditions include spaceflight-induced immune dysregulation and associated comorbidities, spaceflight-induced muscle atrophy, spaceflight orthostatic intolerance, spaceflight or microgravity-associated bone loss, as well as dysbiosis, irritable bowel syndrome, inflammatory bowel disease, immune dysregulation, osteoporosis, frailty, skin hypersensitivity, allergic reactions, viral infection, sarcopenia, inflamm-ageing, muscle wasting, metabolic disorders, cardiac atrophy or neurobehavioral abnormalities associated with a low gravity environment or spaceflight.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient. In preferred aspects, the subject is a human patient. The subject can, for example, be an astronaut.

The term “release controlling excipient” as used herein, refers to an excipient whose primary function is to modify the duration or place of release of the active substance from a dosage form as compared with a conventional immediate release dosage form. The term “non-release controlling excipient” as used herein, refers to an excipient whose primary function do not include modifying the duration or place of release of the active substance from a dosage form as compared with a conventional immediate release dosage form.

The term “substantially pure” as used herein in reference to a given oligosaccharide means that the oligosaccharide is substantially free from other biological macromolecules. The substantially pure oligosaccharide is at least 75% (e.g., at least 80, 85, 95, or 99%) pure by dry weight. Purity can be measured by any appropriate standard method, for example, by column chromatography, polyacrylamide gel electrophoresis, or HPLC analysis.

The terms “treat”, “treating” and “treatment,” as used herein, refers to ameliorating symptoms associated with a disease, condition, or disorder (e.g., a disease, condition or disorder associated with spaceflight or low gravity environment), including inhibiting the progress of the disease or disorder (e.g., a disease, condition or disorder associated with spaceflight or low gravity environment), reducing the severity of the disease or disorder (e.g., a disease, condition or disorder associated with spaceflight or low gravity environment), preventing or delaying the onset of the disease, condition or disorder symptoms, and/or lessening the severity or frequency of symptoms of the disease, condition, or disorder.

Stem cell: The term “stem cell” has not only an autonomously replicating ability, but also a characteristic capable of differentiating into various cells by its multi-potency property, when an appropriate signal is provided if needed under the influence of the environment in which a cell is located, and is comprised in adipose, bone marrow, cord blood and placenta and the like. The stem cell of the present invention may be an autologous or allogenic derived stem cell, and may be derived from any type of animals including humans and non-human mammals.

The invention provides means of preventing microgravity associated bone loss. In some embodiments, enriched populations of stem cells or stem cell precursors are used to promote the growth and maturation osteoblasts. In some embodiments, bone tissue is contacted by an enriched population of stem cells or stem cell precursors wherein the stem cells or stem cell precursors promote the growth and maturation osteoblasts tissue as well as reversing senescence. In some embodiments, after contact with the bone tissue, the stem cells or stem cell precursors migrate to bone compartments. In some embodiments, the bone tissue is contacted by an enriched population of stem cells or stem cell precursors in vivo. In some embodiments, in vivo administration includes, but is not limited to, localized injection (e.g., catheter administration or direct intra-bone injection), systemic injection, intravenous injection, intrauterine injection, and parenteral administration. In some embodiments, the bone tissue is contacted by an enriched population of stem cells or stem cell precursors ex vivo. In some embodiments, ex vivo contact includes, but is not limited to, direct injection of bone tissue, aggregation with intact or dissociated bone tissue, and co-culture with bone tissue. In some embodiments, the contacted ex vivo bone tissue is cultured with stem cell or stem cell precursors and then transplanted or implanted into a subject's ovaries or surrounding tissues. Methods for transplanting or implanting include, but are not limited to, engraftment onto ovary, injection or engraftment of tissue into ovary following bone incision, and engraftment into fallopian tube. In some embodiments, the contacted ex vivo bone tissue is cultured and then frozen and stored after growth and maturation of the osteoblast bone tissue may be any mammalian bone tissue. In some embodiments, the enriched population of stem cells or stem cell precursors and the bone tissue are autologous

Numerous types of regenerative cells, such as stem cells may be used for the practice of the invention including. The underlying theme of the invention teaches the use of cells with stem cell-like properties for the treatment of bone loss caused by microgravity. Specific properties of stem cells that are suitable for use in practicing the current invention are:

a) ability to both increase endothelial function, as well as induce neoangiogenesis which helps overcome fibrosis; b) ability to prevent atrophy, as well as to differentiate into functional bone and/or bone tissue; and c) ability to induce local resident stem/progenitor cells to proliferate through secretion of soluble factors, as well as via membrane bound activities.

In one embodiment of the invention, stem cells are collected from an autologous patient, expanded ex vivo, and reintroduced into said patient at a concentration and frequency sufficient to cause therapeutic benefit in osteoporosis. Said stem cells are selected for ability to cause: neoangiogenesis, prevention of tissue atrophy, and regeneration of functional tissue. Stem cells chosen may be selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

According to the teachings of the invention, when selecting stem cells for use in the practice of the current disclosure, several factors must be taken into consideration, such as: ability for ex vivo expansion without loss of therapeutic activity, ease of extraction, general potency of activity, and potential for adverse effects. Ex vivo expansion ability of stem cells can be measured using typical proliferation and colony assays known to one skilled in the art, while identification of therapeutic activity depends on functional assays that test biological activities such as: ability to support endothelial function, ability to protect granulosa cells from degeneration/atrophy, and, ability to inhibit bone cortex tissue from atrophy/degeneration. Assessment of therapeutic activity can also be performed using surrogate assays which detect markers associated with a specific therapeutic activity. Such markers include CD34 or CD133, which are associated with stem cell activity and ability to support angiogenesis and osteogenesis. Other assays useful for identifying therapeutic activity of stem cell populations for use with the current invention include evaluation of production of factors associated with the therapeutic activity desired. For example, identification and quantification of production of FGF, VEGF, angiopoietin, or other such angiogenic molecules may be used to serve as a guide for approximating therapeutic activity in vivo. Additionally, secretion of factors that inhibit smooth muscle atrophy or follicular dysfunction may also be used as a marker for identification of cells that are useful for practicing the current invention.

For use in the context of the present invention, embryonic stem cells possess certain desirable properties, such as the ability to differentiate to almost every cell comprising the host. Additionally, embryonic stem cells secrete numerous factors capable of inhibiting the process of osteoblast fibrosis or bone atrophy. Unfortunately, certain drawbacks exist that limit the utility of this cell type for widespread therapeutic implementation. The potential for carcinogenicity is apparent in that human embryonic stem cells administered to immunocompromised mice leads to formation of teratomas. Accordingly, for use in the current invention, embryonic stem cells need to be either differentiated into a stem cell, or a progenitor cell that is not capable of forming tumors.

Cells useful the practice of the current invention should not differentiate in a substantial amount in an uncontrolled manner or into tissue which is pathological to the patient's health. Although several technologies are currently being tested for selecting embryonic stem cells that do not cause teratomas, these methods are still in their infancy. Therefore, one method of utilizing embryonic stem cells for the practice of this invention is to encapsulate said embryonic stem cells, or place said cells into a permeable barrier so as to allow for secretion of therapeutic factors elaborated by said cells without the risk of causing cancer or undesired tissue growth. Such encapsulated cells may be administered subcutaneously, intramuscularly, intra-omentally, or into the ovary.

The use of said encapsulation technology has been successful in “semi-isolating” cells with therapeutic potential from the body, for examples of this the practitioner of the invention is referred to work on microencapsulation of islets for treatment of diabetes, in which cases xenogeneic islets are used, or other systems of therapeutic cellular xenograft therapy. Said encapsulated cells may be administered systemically, or in a preferred embodiment locally in an area proximal to penile circulation, such as the fat tissue adjacent to the pudendal artery. Alternatively, encapsulated cells may be placed in a removable chamber in subcutaneous tissue similarly to the one described in U.S. Pat. No. 5,958,404. The advantages of using a removable chamber is that administration of cell therapy is not a permanent intervention and may be withdrawn upon achievement of desired therapeutic effect, or at onset of adverse effects. Another embodiment of the current invention is the use of embryonic stem cell supernatant as a therapy for osteoporosis associated with hormonal imbalances such as bone loss in space. Specific embodiments include identification of substantially purified fractions of said supernatant capable of inducing endothelial cell proliferation, smooth muscle regeneration, and/or neuronal cell proliferation/survival. Identification of such therapeutically active fractions may be performed using methods commonly known to one skilled in the art, and includes separation by molecular weight, charge, affinity towards substrates and other physico-chemical properties.

In one particular embodiment, supernatant of embryonic stem cell cultures is harvested substantially free from cellular contamination by use of centrifugation or filtration. Supernatant may be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of embryonic stem cell supernatant may be passed through each cartridge before elution. After washing the cartridges material adsorbed is eluted with 3 ml methanol, evaporated under a stream of nitrogen, redissolved in a small volume of methanol, and stored at 4.degree. C. Before testing the eluate for activity in vitro, the methanol is evaporated under nitrogen and replaced by culture medium. Said C18 cartridges are used to adsorb small hydrophobic molecules from the embryonic stem cell culture supernatant, and allows for the elimination of salts and other polar contaminants. It may, however be desired to use other adsorption means in order to purify certain compounds from the embryonic stem cell supernatant. Said concentrated supernatant may be assessed directly for biological activities useful for the practice of this invention, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Embryonic stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

For the practice of the invention, the practitioner is referred to the numerous methods of generating embryonic stem cells that are known in the art. Patents describing the generation of embryonic stem cells include U.S. Pat. No. 6,506,574 to Rambhatla, U.S. Pat. No. 6,200,806 to Thomson, U.S. Pat. No. 6,432,711 to Dinsmore, and U.S. Pat. No. 5,670,372 to Hogan. In one embodiment of the invention, embryonic stem cells are differentiated into endothelial progenitor cells in vitro, followed by administration to a patient in need of therapy at a concentration and frequency sufficient to ameliorate or cure ED. Differentiation into endothelial progenitors may be

performed by several means known in the art [74]. One such means includes generation of embryoid bodies through growing human embryonic stem cells in a suspension culture. Said embryoid bodies are subsequently dissociated and cells expressing endothelial progenitor

markers are purified [75]. Purification of endothelial cells from embryoid bodies can be performed using, of example, selection for PECAM-1 expressing cells. Purified cells can be expanded in culture and used for injection. Another alternative method of generating endothelial progenitors from embryonic stem cells involves removing media from embryonic stem cells a period of time after said embryonic stem cells are plated and replacing said media with a media containing endothelial-differentiating factors. For example, after plating of embryonic stem cells for a period between 6 and 48 hours, but more preferably between 20 and 24 hours, the original media in which embryonic stem cells were cultured is washed off the cells and endothelial cell basal medium-2 (EBM2), with 5% fetal calf serum, VEGF, bFGF, IGF-1, EGF, and ascorbic acid is added to the cells. This combination is commercially available (EGM2-MV Bullet Kit; Clonetics/BioWhittaker, Walkersville, MD). By culturing the embryonic stem cells for 20-30 days in the EGM2 medium, with changing of media every 3 to 5 days, a population of endothelial progenitors can be obtained. For such cells to be useful in the practice of the present invention, functionality of said endothelial precursors, and their differentiated progeny must be assessed. Methods of assessing endothelial function include testing their ability to produce and respond to NO, as well as ability to form cord-like structures in Matrigel, and/or form blood

vessels when injected into immunocompromised mice [76]. Endothelial cells, or endothelial precursor cells, generated from embryonic stem cells may be administered to the patient in an injection solution, which may be saline, mixtures of autologous plasma together with saline, or various concentrations of albumin with saline. Ideally pH of the injection solution is from about 6.4 to about 8.3, optimally 7.4. Excipients may be used to bring the solution to isotonicity such as, 4.5% mannitol or 0.9% sodium chloride, pH buffers with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. Injection can be performed systemically, with the goal of injected cells homing to penile tissues associated with ED, or alternatively administration may be local, via intracavernosal administration. In variations of the invention where endothelial progenitors/endothelial cells are administered systemically, the local administration of an endothelial progenitor/endothelial cell chemoattractant factor may be used in order to increase the number of cells homing to the area of need. Said chemoattractant factors may include SDF-1 and/or VEGF, various isoforms thereof and small molecule agonists of the VEGFR-1 and/or VEGFR2, and/or CXCR4. Localization of said chemotactic factors to the area causative of ED may be performed using agents such as fibrin glue or certain delivery polymers known to one who is skilled in the art, these may include: polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol. Acceptable carriers, excipients, or stabilizers are also contemplated within the current invention, said carriers, excipients and stabilizers being relatively nontoxic to recipients at the dosages and concentrations employed, and may include buffers such as phosphate, citrate, and other organic acids; antioxidants including ascorbic acid, n-acetylcysteine, alpha tocopherol, and methionine; preservatives such as hexamethonium chloride; octadecyldimethylbenzyl ammonium chloride; benzalkonium chloride; phenol, benzyl alcohol, or butyl; alkyl parabens such as methyl or propyl paraben; catechol; resorcinol; cyclohexinol; 3-pentanol; and me-cresol); low molecular weight polypeptides; proteins, such as gelatin, or non-specific immunoglobulins; amino acids such as glycine, glutamine, asparagine, histidine, arginine, or lysine; monosaccharides, disaccharides, and other carbohydrates including glucose, mannose, or dextrins; chelating agents such as. EDTA; sugars such as sucrose, mannitol, trehalose, or sorbitol; salt-forming counter-ions such as sodium; metal complexes. Chemoattraction of cells with stem cell-like properties has been described in US patent application #20060003312 to Blau.

In another embodiment, embryonic stem cells are induced to differentiate into the neuronal lineage in vitro, prior to administration into a patient suffering from osteoporosis. Said differentiation may be performed by alteration of culture conditions, such as growing embryonic stem cells in suspension culture so as to allow formation of embryoid bodies, and collecting the follicle differentiated cells from said embryoid bodies, followed by in vitro expansion. Collection of the follicle differentiated cells may be performed through selective isolation of cells expressing makers associated with the neuronal lineage. In another embodiment embryonic stem cells are differentiated into a desired phenotype microencapsulated so as to retain viability and ability to produce growth factors, while at the same time escaping immune mediated killing. This may be accomplished using known microencapsulation methods described in the art, such as described in U.S. Pat. No. 7,041,634 to Weber et al, or US Patent Application # 20040136971 to Scharp et al. Additionally, embryonic stem cells may be irradiated either prior to, or subsequent to, encapsulation so as to block ability to proliferate while retaining growth factor producing activity. In another embodiment embryonic stem cells are grown on the outside of a hollow-fiber filter which is connected to a continuous extracorporeal system. Said hollow-fiber system contains pores in the hollow fiber of sufficient size so has to allow exchange of proteins between circulating blood cells and cultured cells on the outside of the hollow fibers, without interchange of host cells with the embryonic stem cells.

In another embodiment of the invention, cord blood stem cells are administered systemically into a patient suffering from osteoporosis associated with space travel. Said cord blood stem cells may be administered as a heterogenous population of cells by the administration of cord blood mononuclear cells.

Said cells may be isolated according to many methods known in the art. In one particular method, cord blood is collected from fresh placenta and mononuclear cells are purified by centrifugation using a density gradient such as Ficoll or Percoll, in another method cord blood mononuclear cells are isolated from contaminating erythrocytes and granulocytes by the Hetastarch with a 6% (wt/vol) hydroxyethyl starch gradient. Cells are subsequently washed to remove contaminating debris, assessed for viability, and administered at a concentration and frequency sufficient to induce therapeutic benefit.

In another embodiment of the invention, cord blood stem cells are fractionated and the fraction with enhanced therapeutic activity is administered to the patient. Enrichment of cells with therapeutic activity may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the invention, it will be useful to select cord blood stem cells with a higher proliferative ability, whereas in other situations, a lower proliferative ability may be desired. In some embodiments of the invention, cells are directly injected into the area of need, such as in bone loss in which case it will be desirable for said stem cells to be substantially differentiated, whereas in other embodiments, cells will be administered systemically and it this case with may be desirable for the administered cells to be less differentiated, so has to still possess homing activity to the area of need.

In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342.

Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for ED, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

Certain desired activities can be endowed onto said cord blood stem cells prior to administration into the patient. In one specific embodiment cord blood cells may be “activated” ex vivo by a brief culture in hypoxic conditions in order to upregulate nuclear translocation of the HIF-1 transcription factor and endow said cord blood cells with enhanced angiogenic potential. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, can be performed prior to administration of said cord blood cells into the patient. Assessment methods are known in the art and include measurement of angiogenic factors, ability to support viability and activity of cells associated with erectile function, as well as ability to induce regeneration of said cellular components associated with erectile function.

In addition to induction of hypoxia, other therapeutic properties can be endowed unto cord blood stem cells through treatment ex vivo with factors such as de-differentiating compounds, proliferation inducing compounds, or compounds known to endow and/or enhance cord blood cells to possess properties useful for the practice of the current invention. In one embodiment cord blood cells are cultured with an inhibitor of the enzyme GSK-3 in order to enhance expansion of cells with pluripotent characteristics while not increasing the rate of differentiation. In another embodiment, cord blood cells are cultured in the presence of a DNA methyltransferase inhibitor such as 5-azacytidine in order to endow a “de-differentiation” effect. In another embodiment cord blood cells are cultured in the presence of a differentiation agent that skews said cord blood stem cells to generate enhance numbers of cells which are useful for treatment of space travel associated osteoporosis after said cord blood cells are administered into a patient. For example, cord blood cells may be cultured in estrogen for a brief period so that subsequent to administration, an increased number of follicular cells generated in the patient in need thereof.

In contrast to cord blood stem cells, placental stem cells may be purified directly from placental tissues, said tissues including the chorion, amnion, and villous stroma. In another embodiment of the invention, placental tissue is mechanically degraded in a sterile manner and treated with enzymes to allow dissociation of the cells from the extracellular matrix. Such enzymes include, but not restricted to trypsin, chymotrypsin, collagenases, elastase and/or hylauronidase. Suspension of placental cells are subsequently washed, assessed for viability, and may either be used directly for the practice of the invention by administration either locally or systemically. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations, it will be desirable to expand cells before introduction into the human body. Expansion can be performed by culture ex vivo with specific growth factors [80, 81]. The various embodiments of the invention described above for cord blood and embryonic stem cells can also be applied for placental stem cells.

In some embodiments of the invention, administration of “platelet rich plasma” is performed in order to augment regenerative activities of stem cells. Administration may be locally with the stem cells or may be in a place different from the stem cells. “Platelet rich plasma” (PRP) as described herein is a blood plasma that has been enriched with platelets. As a concentrated source of autologous platelets, PRP contains and releases several different growth factors and other cytokines that stimulate healing of bone and soft tissue. Components of PRP may include but are not limited to platelet-derived growth factor, transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, and combinations thereof. PRP may be prepared by collection of the patient's whole blood (that is anticoagulated with citrate dextrose) before undergoing two stages of centrifugation designed to separate the PRP aliquot from platelet-poor plasma and red blood cells. In humans, a typical baseline blood platelet count may range from about 150,000 to about 450,000 platelets per.mu.l of blood, or about 200,000 platelets per.mu.l of blood. Therapeutic PRP may concentrate platelets in plasma by about five-fold. As such, PRP platelet count in PRP may range from about 750,000 to about 2.25.times.10.sup.6 platelets per .mu.1 of PRP, or about 1.times.10.sup.6 platelets per .mu.1 of blood. The PRP may then be used to prepare human platelet lysate. Compositions of the present disclosure may comprise platelet plasma compositions from PRP, HPL, or combinations thereof, and either platelet plasma composition may be used to regenerate bone tissue for augmentation of fertility. Further, the platelet plasma composition may be used with or without concentrated bone marrow (BMAC). By way of example, when administered into bone tissue, about 0.05 to about 2.0 cc of platelet plasma composition may be used. Platelets are non-nucleated blood cells that as noted above are found in bone marrow and peripheral blood. In various embodiments of the present invention, the platelet plasma composition may be obtained by sequestering platelets from whole blood or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires bone tissue regeneration. In some embodiments the lysate will act more rapidly than the PRP (or platelet poor plasma from bone marrow). Human platelet lysate may be formed from but not limited to PRP, pooled platelets from humans, and cultured megakaryocytes from stem cell expansion technology. In some embodiments, HPL is from a commercial source. In some embodiments, HPL is prepared in the laboratory from platelet rich plasma (PRP), pooled platelets from humans, or cultured megakaryocytes from stem cell expansion technology. Notably, platelet poor plasma that is derived from bone marrow has a greater platelet concentration than platelet rich plasma from blood, also known as platelet poor/rich plasma (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow). In various embodiments, the platelet plasma composition, which may or may not be in the form of a lysate, may serve one or more of the following functions: (1) to release/provide growth factors and cytokines for tissue regeneration; (2) to reduce inflammation; (3) to attract/mobilize cell signaling; (4) to initiate repair of damaged/atrophied bone tissue through fibroblast growth factors (FGF); (5) to stabilize extracellular matrix in the ovary; (6) to stimulate maturation of immature oocytes; (7) to stimulate revascularization of fibrotic tissue; and (8) to stimulate oocyte receptivity to spermatozoa. Additionally, by combining platelet therapy with stem cells, there can be synergy with respect to augmentation of fertility. In some embodiments in which the lysate is used, the cytokines may be concentrated in order to optimize their functional capacity. Concentration may be accomplished in two steps. First, blood may be obtained and concentrated to a volume that is of what it was before concentration. Devices that may be used include but are not limited to a hemofilter or a hemo-concentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing cc of peripheral blood, 40-250 cc of peripheral blood, or 60-100 cc of peripheral blood. The amount of blood that one should draw will depend on the extent of bone tissue degeneration. In some embodiments, a method of generation of said PRP may be used according to U.S. Pat. No. 9,011,929, which is incorporated by reference herein in its entirety. In essence, a method may comprise separating PRP from whole blood by collecting whole blood from an animal or patient into a vacuum test tube containing sodium citrate, and primarily centrifuging the collected whole blood; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid; and collecting the PRP concentrated in a bottom layer by another blunt needle; mixing the PRP collected from the separating step with a calcium chloride solution by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at room temperature before mixing; and mixing the mixture of the PRP and the calcium chloride solution with the type I collagen, in an opaque phase, four times by another three-way connector. In an exemplary embodiment of the disclosure, a method may comprise separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector. The term “platelet-rich plasma” or “PRP” as used herein is a broad term which is used in its ordinary sense and is a concentration of platelets greater than the peripheral blood concentration suspended in a solution of plasma, or other excipient suitable for administration to a human or non-human animal including, but not limited to, isotonic sodium chloride solution, physiological saline, normal saline, dextrose 5% in water, dextrose 10% in water, Ringer solution, lactated Ringer solution, Ringer lactate, Ringer lactate solution, and the like. PRP compositions may be an autologous preparation from whole blood taken from the subject to be treated or, alternatively, PRP compositions may be prepared from a whole blood sample taken from a single donor source or from whole blood samples taken from multiple donor sources. In general, PRP compositions comprise platelets at a platelet concentration that is higher than the baseline concentration of the platelets in whole blood. In some embodiments, PRP compositions may further comprise WBCs at a WBC concentration that is higher than the baseline concentration of the WBCs in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration. In some embodiments, PRP compositions comprise elevated concentrations of platelets and WBCs and lower levels of RBCs and hemoglobin relative to their baseline concentrations. In some embodiments of PRP composition, only the concentration of platelets is elevated relative to the baseline concentration. Some embodiments of PRP composition comprise elevated levels of platelets and WBCs compared to baseline concentrations. In some embodiments, PRP compositions comprise elevated concentrations of platelets and lower levels of neutrophils relative to their baseline concentrations. Some embodiments of PRP composition comprise elevated levels of platelets and neutrophil-depleted WBCs compared to their baseline concentrations.

In some embodiments of PRP, the ratio of lymphocytes and monocytes to neutrophils is significantly higher than the ratios of their baseline concentrations. The PRP formulation may include platelets at a level of between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood comprises at least 140,000 to 150,000 platelets and up to 400,000 to 500,000 platelets. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter. The WBC concentration is typically elevated in PRP compositions. For example, the WBC concentration may be between about 1.01 and about 2 times the baseline, about 2 and about 3 times the baseline, about 3 and about 4 times the baseline, about 4 and about times the baseline, about 5 and about 6 times the baseline, about 6 and about 7 times the baseline, about 7 and about 8 times the baseline, about 8 and about 9 times the baseline, about 9 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000; about 10,000 and about about 15,000 and about 20,000; about 20,000 and about 30,000; about 30,000 and about about 50,000 and about 75,000; and about 75,000 and about 100,000. Among the WBCs in the PRP composition, the concentrations may vary by the cell type but, generally, each may be elevated.

In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration.

The concentrations of eosinophils in the PRP composition may be less than baseline, about 1.5 times baseline, about 2 times baseline, about 3 times baseline, about 5 times baseline, or higher. In whole blood, the lymphocyte count is typically between 1,300 and 4,000 cells per microliter, but in other examples, the lymphocyte concentration may be between about and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter.

In certain variations, the PRP composition may contain a specific concentration of neutrophils. The neutrophil concentration may vary between less than the baseline concentration of neutrophils to eight times than the baseline concentration of neutrophils. In some embodiments, the PRP composition may include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels are depleted to 1% or less than that found in whole blood. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, about 5,000 to about 20,000 per microliter, about 20,000 to about 40,000 per microliter, or about to about 60,000 per microliter. In some embodiments, neutrophils are eliminated or substantially eliminated. Means to deplete blood products, such as PRP, of neutrophils is known and discussed in U.S. Pat. No. 7,462,268, which is incorporated herein by reference. Several embodiments are directed to PRP compositions in which levels of platelets and white blood cells are elevated compared to whole blood and in which the ratio of monocytes and/or lymphocytes to neutrophils is higher than in whole blood. The ratio of monocytes and/or lymphocytes to neutrophils may serve as an index to determine if a PRP formulation may be efficaciously used as a treatment for a particular disease or condition. PRP compositions where the ratio of monocytes and/or lymphocytes to neutrophils is increased may be generated by either lowering neutrophils levels, or by maintaining neutrophil levels while increasing levels of monocytes and/or lymphocytes. Several embodiments relate to a PRP formulation that contains 1.01 times, or higher, baseline platelets in combination with a 1.01 times, or higher, baseline white blood cells with the neutrophil component depleted at least 1% from baseline. In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women. In some embodiments, PRP compositions contain increased concentrations of growth factors and other cytokines. In several embodiments, PRP compositions may include increased concentrations of one or more of: platelet-derived growth factor, transforming growth factor beta, fibroblast growth factor, insulin-like growth factor, insulin-like growth factor 2, vascular endothelial growth factor, epidermal growth factor, interleukin-8, keratinocyte growth factor, and connective tissue growth factor.

In some embodiments, the platelets collected in PRP are activated by thrombin and calcium chloride to induce the release of these growth factors from alpha granules. In some embodiments, a PRP composition is activated exogenously with thrombin and/or calcium to produce a gel that can be applied to an area to be treated. The process of exogenous activation, however, results in immediate release of growth factors. Other embodiments relate to activation of PRP via in vivo contact with collagen containing tissue at the treatment site. The in vivo activation of PRP results in slower growth factor release at the desired site. In certain embodiments of the invention, the PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the tissue damage and/or the MI, the availability of prepared PRP, or any suitable combination of factors. Any suitable amount of blood may be collected. For example, about 1 cc to about 150 cc of blood or more may be drawn. More specifically, about 27 cc to about 110 cc or about 27 cc to about 55 cc of blood may be withdrawn. In some embodiments, the blood may be collected from a patient who may be presently suffering, or who has previously suffered from, connective tissue damage and/or an MI. PRP made from a patient's own blood may significantly reduce the risk of adverse reactions or infection. In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area. The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume. In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained. As the PRP composition comprises activated platelets, active agents within the platelets are released. These agents include, but are not limited to, cytokines (e.g., IL-1B, IL-6, TNF-A), chemokines (e.g., ENA-78 (CXCL8), IL-8 (CXCL8), MCP-3 (CCL7), MIP-1A (CCL3), NAP-2 (CXCL7), PF4 (CXCL4), RANTES (CCLS)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-.beta. 1, 2, and 3, and VEGF). Said PRP may be used to treat autologous regenerative cells prior to administration of said cells for stimulation of ovary regeneration and/or prevention of immunologically mediated abortions.

One type of autologous regenerative cells are adipose stromal vascular fraction cells. Said stromal vascular fraction cells are obtained by the following steps; a) Using aseptic technique and with local anesthesia, the infra-umbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37.degree. C. shaking water bath for one hour then centrifuged for 5 min at 300 rcf; g) Following centrifugation, the stromal cells are resuspended within Isolyte in separate sterile 50 mL centrifuge tubes; h) The tubes are centrifuged for 5 min. at 300 rcf and the Isolyte is removed, leaving cell pellet; i) The pellets are resuspended in 40 ml of Isolyte, centrifuged again for 5 min at 300rcf. The supernatant is again be removed; j) The cell pellets are combined and filtered through 100.quadrature.m cell strainers into a sterile 50 ml centrifuge tube and centrifuged for 5 min at 300rcf and the supernatant removed, leaving the pelleted adipose stromal cells. Means of combining PRP and SVF are known in the literature and incorporated by reference.

In some embodiments, the neutrophils are depleted by at least 5%, in some embodiments, the neutrophils are depleted by at least 10%, in some embodiments, the neutrophils are depleted by at least 15%, in some embodiments, the neutrophils are depleted by at least 20%, in some embodiments, the neutrophils are depleted by at least 25%, in some embodiments, the neutrophils are depleted by at least 30%, in some embodiments, the neutrophils are depleted by at least 35%, in some embodiments, the neutrophils are depleted by at least 40%, in some embodiments, the neutrophils are depleted by at least 45%, in some embodiments, the neutrophils are depleted by at least 50%, in some embodiments, the neutrophils are depleted by at least 55%, in some embodiments, the neutrophils are depleted by at least 60%, in some embodiments, the neutrophils are depleted by at least 65%, in some embodiments, the neutrophils are depleted by at least 70%, in some embodiments, the neutrophils are depleted by at least 75%, in some embodiments, the neutrophils are depleted by at least 80%, in some embodiments, the neutrophils are depleted by at least 85%, in some embodiments, the neutrophils are depleted by at least 90%, in some embodiments, the neutrophils are depleted by at least 95%, in some embodiments, the neutrophils are depleted by at least 95%. In some embodiments, the neutrophils in the platelet rich plasma are substantially removed. Administration of PRP intra-ovarially may be performed using methods known in the art. Exemplary publications, which are incorporated by reference for guidance in the practice of the invention are provided. In some embodiments of the invention, autologous regenerative cells such as adipose stromal vascular fraction cells, and/or bone marrow mononuclear cells are administered together with platelet rich plasma and/or platelet lysate.

Bone marrow stem cells may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest”. Said aspirate is used as a starting material for purification of cells with ability to prevent osteoporosis induced by space travel. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of bone loss. The various embodiments of the invention described above for cord blood and embryonic stem cells can also be applied for bone marrow stem cells.

Amniotic fluid is routinely collected during amniocentesis procedures. One method of practicing the current invention is utilizing amniotic fluid derived stem cells for treatment of space travel associated bone loss. In one embodiment amniotic fluid mononuclear cells are utilized therapeutically in an unpurified manner. Said amniotic fluid stem cells are administered either locally or systemically in a patient suffering from bone loss associated with space travel. In other embodiments amniotic fluid stem cells are substantially purified based on expression of markers such as SSEA-3, SSEA4, Tra-1-60, Tra-1-81 and Tra-2-54, and subsequently administered. In other embodiments cells are cultured, as described in US patent application #20050054093, expanded, and subsequently infused into the patient. Amniotic stem cells are described in the following references. One particular aspect of amniotic stem cells that makes them amenable for use in practicing certain aspects of the current invention is their bi-phenotypic profile as being both mesenchymal and neural progenitors.

A wide variety of stem cells are known to circulate in the periphery. These include multipotent, pluripotent, and committed stem cells. In some embodiments of the invention mobilization of stem cells is induced in order to increase the number of circulating stem cells, so that harvesting efficiency is increased. Said mobilization allows for harvest of cells with desired properties for practice of the invention without the need to perform bone marrow puncture. A variety of methods to induce mobilization are known. Methods such as administration of cytotoxic chemotherapy, for example, cyclophosphamide or 5-fluoruracil are effective but not preferred in the context of the current invention due to relatively unacceptable adverse events profile. Suitable agents useful for mobilization include: granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), thrombopoietin (TPO), interleukin-11 (IL-11), insulin-like growth factor-1 (IGF-1), megakaryocyte growth and development factor (MGDF), nerve growth factor (NGF), hyperbaric oxygen, and 3-hydroxy-3-methyl glutaryl coenzyme A (HMG CoA)reductase inhibitors. The various embodiments of the invention described above for cord blood and embryonic stem cells can also be applied for circulating peripheral blood stem cells.

In a preferred embodiment, donors (either autologous or allogeneic) are mobilized by administration of G-CSF (filgrastim: neupogen) at a concentration of 10 ug/kg/day by subcutaneous injection for 2-7 days, more preferably 4-5 days. Peripheral blood mononuclear cells are collected using an apheresis device such as the AS104 cell separator (Fresenius Medical). 1-40×109 mononuclear cells are collected, concentrated and injected into the area of penile flow occlusion in an intramuscular manner. Alternatively, cells may be injected systemically, or in an area proximal to the region of penile blood flow occlusion. Identification of such occlusion is routinely known in the art and includes the use of penile ultrasonometry. Variations of this procedure may include steps such as subsequent culture of cells to enrich for various populations known to possess angiogenic and/or neurogenic, and/or anti-atrophy Additionally cells may be purified for specific subtypes before and/or after culture. Treatments can be made to the cells during culture or at specific time points during ex vivo culture but before infusion in order to generate and/or expand specific subtypes and/or functional properties.

In one embodiment mesenchymal cells are generated through culture. For example, U.S. Pat. No.5,486,359 describes methods for culturing such and expanding mesenchymal stem cells, as well as providing antibodies for use in detection and isolation. U.S. Pat. No. 5,942,225 teaches culture techniques and additives for differentiation of such stem cells which can be used in the context of the present invention to produce increased numbers of cells with angiogenic and/or follicogenic capability. Although U.S. Pat. No. 6,387,369 teaches use of mesenchymal stem cells for regeneration of cardiac tissue, we believe that in accordance with published literature stem cells generated through these means are actually angiogenically potent and therefore may be utilized in the context of the current invention for treatment/amelioration of bone loss. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1.

Mesenchymal stem cells are classically obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue, placenta, scalp tissue and cord blood. A recent study compared mesenchymal stem cells from bone marrow, cord blood and adipose tissue in terms of colony formation activity, expansion potential and immunophenotype. It was demonstrated that all three sources produced mesenchymal stem cells with similar morphology and phenotype. Ability to induce colony formation was highest using stem cells from adipose tissue and interestingly in contrast to bone marrow and adipose derived mesenchymal cells, only the cord blood derived cells lacked ability to undergo adipocyte differentiation. Proliferative potential was the highest with cord blood mesenchymal stem cells which were capable of expansion to approximately 20 times, whereas cord blood cells expanded an average of 8 times and bone marrow derived cells expanded 5

times [94]. Accordingly, one skilled in the art will understand that mesenchymal stem cells for use with the present invention may be selected upon individual patient characteristics and the end result sought. For example, if autologous mesenchymal stem cells are available in the form of adipocyte-derived cells, it will be useful to utilize this source instead of allogeneic cord-blood derived cells. Alternatively, cord blood derived mesenchymal stem cells may be more advantageous for use in situations where autologous cells are not available, and expansion is sought.

Adipose derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently filtered using a cell strainer, for example a 100 μm nylon cell strainer in order to remove debris. Filtered cells are subsequently centrifuged again at approximately 700-2000 g for 5-15 minutes and resuspended at a concentration of approximately 1×106/cm2 into culture flasks or similar vessels. After 10-20 hours of culture non-adherent cells are removed by washing with PBS and remaining cells are cultured at similar conditions as described above for culture of cord blood derived mesenchymal stem cells. Upon reaching a concentration desired for clinical use, cells are harvested, assessed for purity and administered in a patient in need thereof as described above. The various embodiments of the invention described above for cord blood and embryonic stem cells can also be applied for adipose derived stem cells. Tooth derived stem cells have been recently identified as a source of pluripotent stem cells with ability to differentiate into endothelial, neural, and bone structures. Said pluripotent stem cells have been termed “stem cells from human exfoliated deciduous teeth” (SHED). One of the embodiments of the current invention involves utilization of this novel source of stem cells for the treatment of bone loss. In one embodiment of the invention, SHED cells are administered systemically or locally into a patient with bone loss at a concentration and frequency sufficient for induction of therapeutic effect. SHED cells can be purified and used directly, certain sub-populations may be concentrated, or cells may be expanded ex vivo under distinct culture conditions in order to generate phenotypes desired for maximum therapeutic effect. Growth and expansion of SHED has been previously described by others. In one particular method, exfoliated human deciduous teeth are collected from 7- to 8-year-old children, with the pulp extracted and digested with a digestive enzyme such as collagenase type I. Concentrations necessary for digestion are known and may be, for example 1-5 mg/ml, or preferable around 3 mg/ml. Additionally, dispase may also be used alone or in combination, concentrations of dispase may be 1-10 mg/ml, preferably around 4 mg/ml. Said digestion is allowed to occur for approximately 1 h at 37° C. Cells are subsequently washed and may be used directly, purified, or expanded in tissue culture. The various embodiments of the invention described above for cord blood and embryonic stem cells can also be applied for exfoliated teeth stem cells.

The bulge area of the hair follicle bulge is an easily accessible source of pluripotent mesenchymal-like stem cells. One embodiment of the current invention is the use of hair follicle stem cells for treatment of bone loss. Said cells may be used therapeutically once freshly isolated, or may be purified for particular sub-populations, or may be expanded ex vivo prior to use. Purification of hair follicle stem cells may be performed from cadavers, from healthy volunteers, or from patients undergoing plastic surgery. Upon extraction, scalp specimens are rinsed in a wash solution such as phosphate buffered saline or Hanks and cut into sections 0.2-cm. Subcutaneous tissue is de-aggregated into a single cell suspension by use of enzymes such as dispase and/or collagenase. In one variant, scalp samples are incubated with 0.5% dispase for a period of 15 hours. Subsequently, the dermal sheath is further enzymatically de-aggregated with enzymes such as collagenase D. Digestion of the stalk of the dermal papilla, the source of stem cells is confirmed by visual microscopy. Single cell suspensions are then treated with media containing fetal calf serum, and concentrated by pelletting using centrifugation. Cells may be further purified for expression of markers such as CD34, which are associated with enhanced proliferative ability. In one embodiment of the invention, collected hair follicle stem cells are induced to differentiate in vitro into neural-like cells through culture in media containing factors such as FGF-1, FGF-2, NGF, neurotrophin-2, and/or BDNF. Confirmation of neural differentiation may be performed by assessment of markers such as Muhashi, polysialyated N-CAM, N-CAM, A2B5, nestin, vimentin glutamate, synaptophysin, glutamic acid decarboxylase, serotonin, tyrosine hydroxylase, and GABA. Said neuronal cells may be administered systemically, or locally in a patient suffering from bone loss associated with space travel. The various embodiments of the invention described above for cord blood and embryonic stem cells can also be applied for hair follicle stem cells. Parthenogenically derived stem cells can be generated by addition of a calcium flux inducing agent to activate oocytes, followed by purifying and expanding cells expressing embryonic stem cell markers such as SSEA-4, TRA 1-60 and/or TRA 1-81. Said parthenogenically derived stem cells are totipotent and can be used in a manner similar to that described for embryonic stem cells in the practice of the current invention. The various embodiments of the invention described above for cord blood and embryonic stem cells can also be applied for parthenogenically derived stem cells.

In one embodiment, the mesenchymal cells are infused systemically to treat the bone loss but also to treat the impact of radiation exposure due to particles trapped in the Earth's magnetic field; particles shot into space during solar flares (solar particle events); and galactic cosmic rays, which occurs during space travel. The clinical impact not only helps repair and treat the bone loss but help decrease the damage to the human's bone marrow. The impact of low gravity in a pre-clinical model that is also exposed to irradiation was evaluated using umbilical cord lining mesenchymal cells. The umbilical cord lining cells protected against bone loss at 6 months but also demonstrated less impact to the bone marrow from the radiation exposure compared to the control. This also impacted long term survival.

1. A method of preventing low gravity or zero gravity associated bone density loss comprising of administration of a regenerative cell population.

2. The method of claim 1, wherein the low gravity environment is a low planetary orbit, an interplanetary voyage or inhabiting a planet or moon with gravity less than 1 G.

3. The method of claim 2, wherein the low planetary orbit is selected from the group consisting of: low Earth orbit, low Moon orbit and low Mars orbit.

4. The method of claim 1, wherein said bone density loss is associated with enhancement in osteoclast activity.

5. The method of claim 1, wherein said bone density loss is associated with increased levels of TNF alpha.

6. The method of claim 4, wherein said increase osteoclast activity is associated with enhanced RANK ligand activity as compared to an age-matched healthy control.

7. The method of claim 4, wherein said increased osteoclast activity is associated with enhanced interleukin-8 activity as compared to an age-matched healthy control.

8. The method of claim 4, wherein said increased osteoclast activity is associated with enhanced interleukin-11 activity as compared to an age-matched healthy control.

9. The method of claim 1, wherein said regenerative cell is selected from either alone or in combination from a group comprising of: stem cells, committed progenitor cells, and differentiated cells.

10. The method of claim 9, wherein said stem cells are selected from the group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

11. The method of claim 9, wherein said germinal stem cells express markers selected from the group consisting of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1.

12. The method of claim 9, wherein said reprogrammed stem cells are selected from the group consisting of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

13. The method of claim 12, wherein said DNA demethylating agent is selected from the group consisting of: 5-azacytidine, psammaplin A, and zebularine.

14. The method of claim 12, wherein said histone deacetylase inhibitor is selected from the group consisting of: valproic acid, trichostatin-A, trapoxin A and depsipeptide.

15. The method of claims 1, wherein an antioxidant is administered at a therapeutically sufficient concentration to a patient in need thereof that is selected from the group consisting of: ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, and pycnogenol.

16. The method of claim 1, wherein an NF-kappa B inhibitor is administered prior to, subsequently with, or after administration of regenerative cells.

17. The method of claim 16, wherein said bone targeting technology are quantum dots.

18. The method of claim 1, wherein said regenerative cells are used to treat the bone loss and radiation sickness outside of the earth's ozone layer is also caused by exposure to particles trapped in the Earth's magnetic field.

19. The method of claim 1, wherein said regenerative cells are used to protect and treat the bone loss and radiation sickness outside of the earth's ozone layer is also caused by exposure to particles shot into space during solar flares (solar particle events).

20. The method of claim 1, wherein said regenerative cells are used to protect and treat the bone loss and radiation sickness outside of the earth's ozone layer is also caused by exposure to galactic cosmic rays.
